(19) United States
(12) Patent Application Publication
(10) Pub. No.: US 2017/0340899 A1
(43) Pub. Date: Nov. 30, 2017

(54) RADIOACTIVE STENT

(71) Applicant: BOSTON SCIENTIFIC SCIMED, INC., Maple Grove, MN (US)

(72) Inventors: JOHN A. HINGSTON, Framingham, MA (US); GENE T. STORBECK, Franklin, MA (US)

(21) Appl. No.: 15/603,790

(22) Filed: May 24, 2017

Related U.S. Application Data
(60) Provisional application No. 62/341,414, filed on May 25, 2016.

(51) Int. Cl. A61N 5/10; C09D 7/12; A61F 2/82; A61K 51/12; A61L 31/08; A61L 31/10; B05D 1/40; C09D 183/04

(52) U.S. Cl. CPC A61N 5/1002; B05D 1/40; C09D 7/1216; C09D 183/04; A61K 51/1282; A61L 31/088; A61L 31/10; A61F 2/82; A61F 2210/0095; A61F 2240/001; A61F 2310/00; A61L 2420/06; A61N 2005/1004; A61N 2005/1019; A61L 2420/02

(57) ABSTRACT

This disclosure provides design, material, preparation methods, and use alternatives for medical devices. An example method of preparing a stent comprises applying a coating to a portion of the stent at a medical treatment facility, the coating including a plurality of radioactive elements and a substrate. The plurality of radioactive elements are mixed with the substrate to form a mixture such that the plurality of radioactive elements are dispersed within the substrate prior to the coating being applied on the stent.

### CROSS REFERENCE TO RELATED APPLICATIONS

This application claims the benefit of provisional U.S. Patent Application No. 62/341,414, filed on May 25, 2016, which is hereby incorporated by reference in its entirety.

### TECHNICAL FIELD

The present disclosure pertains to medical devices, and methods for preparing medical devices. More particularly, the present disclosure pertains to elongated intracorporeal medical devices including radioactive elements dispersed in a coating, and methods for manufacturing and using such devices.

### BACKGROUND

Some cancers and neoplasms are easier to treat with radiation than others. Hard-to-reach neoplasms, such as those in the esophagus, intestines and other lumens, may be treated via brachytherapy so as to minimize radiation to adjacent, healthy tissue.

Brachytherapy delivers radiation to small tissue volumes while limiting exposure of healthy tissue. In this regard, the delivered radiation conforms more to the target than any other form of radiation, (including proton therapy) as less normal transient tissue is treated. It features placement of radiation sources, such as small radioactive particles or needles, near or within the target tissue, thus having the advantage over External Beam Radiation Therapy (EBRT) of being more focalized and less damaging to surrounding healthy tissue.

Brachytherapy is a common treatment for esophageal, prostate, and other cancers. Brachytherapy has been used to treat prostate cancer which has been practiced for more than half century. In this situation, very low activity material emitting a low energy is placed next to or within a tumor. Traditionally, these low emitting devices have mostly been left in place permanently except in extraordinary circumstances. It would be desirable to utilize radioactive material in conjunction with interventional medical devices when clinically appropriate, and/or it may be desirable to tailor the delivery of radioactive energy or radioactive sources according to clinical needs. For example, it may be advantageous to couple a radiation source with an expandable stent when clinically necessary and/or it may be advantageous to adjust the position and the activity of the radioactive source on a stent in response to changes in tumor shape and size, carrier position, and other relevant therapeutic factors.

### BRIEF SUMMARY

This disclosure provides design, material, manufacturing method, and use alternatives for medical devices.

An example method of preparing a stent comprises applying a coating to a portion of the stent less than 24 hours prior to implanting the stent into a patient, the coating including a plurality of radioactive elements and a substrate. The plurality of radioactive elements are mixed with the substrate to form a mixture such that the plurality of radioactive elements are dispersed within the substrate prior to the coating being applied on the stent.

Alternatively or additionally to any of the embodiments above, wherein the plurality of radioactive elements include a plurality of microspheres.

Alternatively or additionally to any of the embodiments above, wherein the radioactive elements are selected from the group comprising: Iodine-125, Cesium-13, Palladium-103, Yttrium-90 and Holmium-166.

Alternatively or additionally to any of the embodiments above, wherein the substrate includes silicone.

Alternatively or additionally to any of the embodiments above, wherein the half-life of the radioactive elements is less than or equal to 60 days.

Alternatively or additionally to any of the embodiments above, wherein applying the coating to a portion of the stent includes placing the radioactive elements in a first chamber of an applicator, placing the substrate in a second chamber of the applicator, and utilizing the applicator to mix the radioactive elements and the substrate prior to applying the coating to the stent.

Alternatively or additionally to any of the embodiments above, wherein applying the coating to a portion of the stent further includes placing the mixture of the radioactive elements and the substrate on a base member, and thereafter rolling the stent on the base member such that the mixture is applied to the stent.

Alternatively or additionally to any of the embodiments above, wherein applying the coating to a portion of the stent includes placing the mixture of radioactive elements and the substrate into a reservoir, and thereafter dipping the stent into the reservoir such that the mixture is applied to the stent.

An example applicator for applying a radioactive coating on a stent includes a housing including a plurality of discrete chambers, a mixing tube, and a tip member. The mixing tube has a first end and a second end, wherein the first end is connected to the plurality of discrete chambers of the housing. The tip member is connected to the second end of the mixing tube. A first one of the plurality of chambers of the housing is configured to contain a plurality of radioactive elements and at least a second one of the plurality of chambers of the housing is configured to contain a substrate. The plurality of radioactive elements and the substrate are mixed in the applicator prior to being applied to the stent.

Alternatively or additionally to any of the embodiments above, wherein the plurality of radioactive elements includes at least one microsphere.

Alternatively or additionally to any of the embodiments above, wherein the substrate includes silicone.

Alternatively or additionally to any of the embodiments above, wherein the radioactive elements are selected from the group comprising: Iodine-125, Cesium-13, Palladium-103, Yttrium-90 and Holmium-166.

Alternatively or additionally to any of the embodiments above, wherein the plurality of chambers further includes a third chamber containing an initiator, wherein the initiator is configured to cure the substrate.

Alternatively or additionally to any of the embodiments above, wherein the half-life of the radioactive elements is less than or equal to 60 days.

An example kit for preparing a radioactive stent at a medical treatment facility includes a stent and an applicator. The applicator includes a housing including a plurality of discrete chambers. A first one of the plurality of discrete chambers of the applicator is configured to contain a radioactive material. The applicator is configured to apply the radioactive material to the stent prior to implanting the stent within a patient.

Alternatively or additionally to any of the embodiments above, wherein the radioactive material includes a plurality of radioactive microspheres.

Alternatively or additionally to any of the embodiments above, wherein the plurality of discrete chambers of the housing further includes a second chamber and a third chamber, wherein the first chamber includes the plurality of radioactive microspheres, the second chamber includes a silicone and the third chamber includes an initiator designed to cure the silicone.

Alternatively or additionally to any of the embodiments above, wherein the applicator is configured to mix the plurality of radioactive microspheres, the silicone and the initiator prior to applying the radioactive material to the stent.

Alternatively or additionally to any of the embodiments above, wherein the radioactive material is applied to the stent such that the plurality of microspheres are uniformly distributed on the stent.

Alternatively or additionally to any of the embodiments above, wherein the half-life of the plurality of microspheres is less than or equal to 60 days.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The disclosure may be more completely understood in consideration of the following detailed description in connection with the accompanying drawings, in which:

FIG. 1 is an example radioactive stent.

FIG. 2 illustrates an example method to prepare a radioactive stent.

FIG. 3 illustrates another example method to prepare a radioactive stent.

FIG. 4 illustrates another example method to prepare a radioactive stent.

FIG. 5 illustrates another example radioactive stent.

FIG. 6 is a cross-sectional view of another example radioactive stent.

FIG. 7 illustrates another example radioactive stent.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Treatment of abnormal tissue growth (e.g. cancer) may be accomplished through a variety of methodologies. For example, treatment of cancer may include the placement and deployment of a stent across the diseased tissue. However, in some instances stenting outcomes may be improved by combining one or more conventional therapies. For example, combining stent placement with radiation therapy may improve cancer treatment outcomes as compared to either stent or radiation therapy alone. Therefore, it may be desirable to utilize materials and/or design a stent that combines traditional stenting with radiation therapy. Some of the examples and methods disclosed herein may include a stent that can deliver radiation therapy.

Stents disclosed herein may treat esophageal cancers. Additionally, the stent may treat other forms of disease (e.g., cancers), including gastrointestinal, pancreatic, colon, tracheal, urethra, ureter, cardiac, brain, breast, bladder, kyphoplasty and peripheral vascular disease, for example. Further, the stents disclosed herein may also be used in excisional cavities in solid and/or hollow organs.

In some instances it may be desirable to direct radioactive energy to a specific portion of a target area (e.g., a particular portion of a target vessel). Creating variations in the delivery of radioactive energy may be accomplished by controlling the spacing and/or distribution (e.g., coverage area) between the radioactive elements. For example, increasing the number of radioactive elements disposed along a given stent may result a more uniform distribution of radioactive elements for a given surface area along the stent. Furthermore, it can be appreciated that a more uniform distribution may result from increasing the total number of radioactive elements in a given stent. In some instances, it may be desirable to design the distribution of radioactive elements along a stent such that the tissue surrounding stent may receive a substantially uniform amount of radioactive energy. As will be described in greater detail below, examples disclosed herein may discuss both a radioactive coating (e.g., film, foil, etc.) which may be applied uniformly to all of or a selected portion of a stent and related processes of preparing such a radioactive stent.

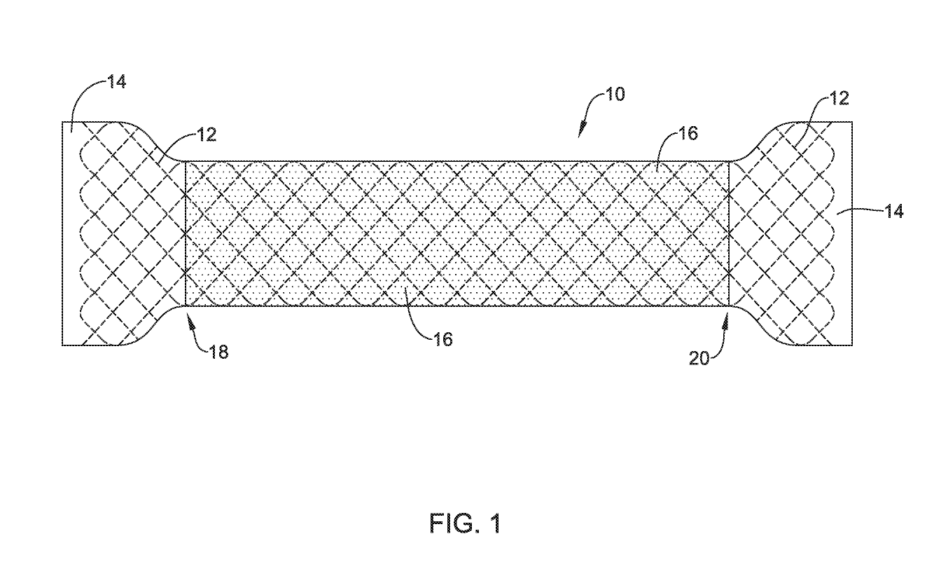

FIG. 1 shows an example radioactive stent 10. Stent 10 may include a plurality of filaments and/or strut members 12 arranged in a variety of different designs and/or geometric patterns. For example, strut members 12 may be a laser cut from a unitary tubular member. In other examples, filaments 12 may be braided, woven, knitted or constructed using a combination of these (or similar) manufacturing techniques. Therefore, numerous designs, patterns and/or configurations for the stent cell openings, strut thicknesses, strut designs, stent cell shapes are contemplated and may be utilized with embodiments disclosed herein.

Further, stent 10 may be delivered to a treatment area via a stent delivery system (not shown). For example, in some instances stent 10 may be a balloon expandable stent. Balloon expandable stents may be manufactured from a single, cylindrical tubular member (e.g., a cylindrical tubular member may be laser cut to form balloon expandable stent 10).

In other instances stent 10 may be a self-expanding stent. A self-expanding stent may be delivered to a treatment area via a self-expanding stent delivery system. It is contemplated that the examples disclosed herein may be utilized with any one of various stent configurations, including, balloon expandable stents, such as a laser cut stent and/or a braided stent, a self-expanding stent, non-expandable stents, or other stents.

Stent filaments 12 disclosed herein may be constructed from a variety of materials. For example, filaments 12 may be constructed from a metal (e.g., Nitinol). In other instances, filaments 12 may be constructed from a polymeric material (e.g., PET). In yet other instances, filaments 12 may be constructed from a combination of metallic and polymeric materials. Additionally, filaments 12 may include a bioabsorbable and/or biodegradable material. Additionally, as shown in FIG. 1, stent 10 may include first and/or second flared end regions.

In some examples, stent 10 may include a covering 14. For example, stent 10 may be partially or fully covered by an elastomeric or non-elastomeric material. Additionally, stent 10 may be partially or fully covered by a polymeric material such as silicone or ePTFE. Further, the covering (e.g., polymer) 14 may span the spaces (e.g., openings, cells) created by the geometric arrangements of filaments 12.

As discussed above, in some examples all or a portion of stent 10 may include a radioactive coating 16. Radioactive coating 16 may constructed by combining one or more substances together to form a compound (e.g., composition, mixture, etc.) capable of being disposed (e.g., placed, distributed, positioned, applied, etc.) on a medical device (e.g., stent) using a variety of processing techniques. For example, radioactive coating 16 may include a radioactive material combined with a substrate. In some instances, the radioactive material may include radioactive “microspheres” (e.g., radioactive microbeads). In some instances, radioactive microspheres may be defined as a radiation source consisting of very small particles (e.g., spheres, balls, etc.) of radioactive material. For example, a plurality of microspheres may bear some resemblance to a fine powder. For purposes of this disclosure, a microsphere may be defined as a particle having a diameter from 1 nanometer to 1 millimeter. However, in some instances, the radioactive material may include radioactive particles having a diameter less than 1 nanometer or greater than 1 millimeter. In some examples, microspheres may include the radioactive material present within radioactive “seeds.”

The radioactive particles or elements, such as microspheres, may include any radioactive material or combinations of various materials such as Iodine-125 (e.g. GE Oncura THINSeed™, IsoAid Advantage™ by IsoAid, Best™ Iodine-125), Palladium-103 (e.g. CivaString™ by CivaTech Technology, Theraseed™ by Theragenics, Best™ Palladium-103), Cesium-131, Gold-198, Iridium-192, Yttrium-90, Holmium-166 and/or Ytterbium-169 or any other variations and/or derivatives thereof. Further, radioactive microspheres may include other types of radioactive material. Additionally, the microspheres may include beta-emitting radionuclides.

The radioactive particles or elements, such as microspheres, (e.g., radioactive material) contemplated in at least some examples herein may include half-life durations that are 365 days or less, 270 days or less, 180 days or less, 90 days or less, 60 days or less, 30 days or less, 20 days or less, 10 days or less, 5 days or less, or shorter time periods. However, while radioactive material having a half-life of less than or equal than 365 days is contemplated, it is also contemplated that some examples herein may utilize radioactive material having a half-life of greater than 365 days.

As discussed above, one example of radioactive coating 16 contemplated herein may be formed of a radiopaque mixture including a radioactive material that is combined (e.g., mixed) with a substrate. In some examples, the “substrate” may be defined as material that is mixed with the radioactive material to form either a heterogeneous material or a homogenous material. In other words, some examples of a radioactive coating contemplated herein include a substance that is formed when solid, radioactive material (e.g., radioactive microspheres) are dispersed (e.g., uniformly or non-uniformly) throughout the substrate. In other examples, solid radioactive material (e.g., radioactive microspheres) may be combined and eventually dissolve within a substrate, forming a homogenous radioactive coating.

In some examples, the substrate may include a plurality of materials combined together. For example, in at least one example contemplated herein, the substrate includes a two-part silicone including one part silicone combined with an initiator or other curing agent. The initiator may be configured to rapidly cure the silicone once the radioactive coating is applied to the stent 10. The radioactive mixture may cure in 24 hours or less, 12 hours or less, 6 hours or less, 120 minutes or less, 60 minutes or less, or 30 minutes or less, in some instances. Examples of substrate materials contemplated herein may include a variety of polymer-based materials. For example, an example substrate may include a self-cure liquid silicone rubber, or other self-cure polymer. Additionally, the substrate material may include a temperature cure silicone rubber and/or a variety of elastomers.

As discussed above, it may be desirable to coat the stent 10 (including filaments 12 and/or covering 14) with the radioactive coating 16. The radioactive coating 16 may be disposed along any portion of the stent 10. For example, FIG. 1 shows the radioactive coating 16 disposed along a portion of the stent 10 from a first end 18 to a second end 20. However, while FIG. 1 illustrates the coating 16 disposed from a first end 18 to a second end 20, it is contemplated that radioactive coating 16 may be disposed along any portion of the stent 10 (including any portion of the luminal surface, abluminal surface and/or other portions of the filaments 12 and/or the covering 14).

The radioactive coating 16 may be applied (e.g., disposed) along the outer surface of the filaments 12. In other instances it may be favorable to ensure that the radioactive coating 16 is positioned on the inner surface of the filaments 12. In yet other examples, it may be desirable to dispose the radioactive coating 16 along a combination of the outer surface and inner surface of the filaments 12. In some instances, the radioactive coating 16 may be applied to a surface of the covering 14, such as an outer surface and/or an inner surface of the covering 14. Additionally, in some instances it may be desirable to apply the radioactive coating 16 to all surfaces of the filaments 12 and/or the covering 14. Application of a more uniform radioactive coating 16 along a specific location of the stent 10 and/or in a specific geometric pattern (e.g., distribution, arrangement, etc.) may minimize the occurrence of “hot spots” (e.g., localized radiation sources) at the tissues contacting the stent 10 near the radioactive coating 16.

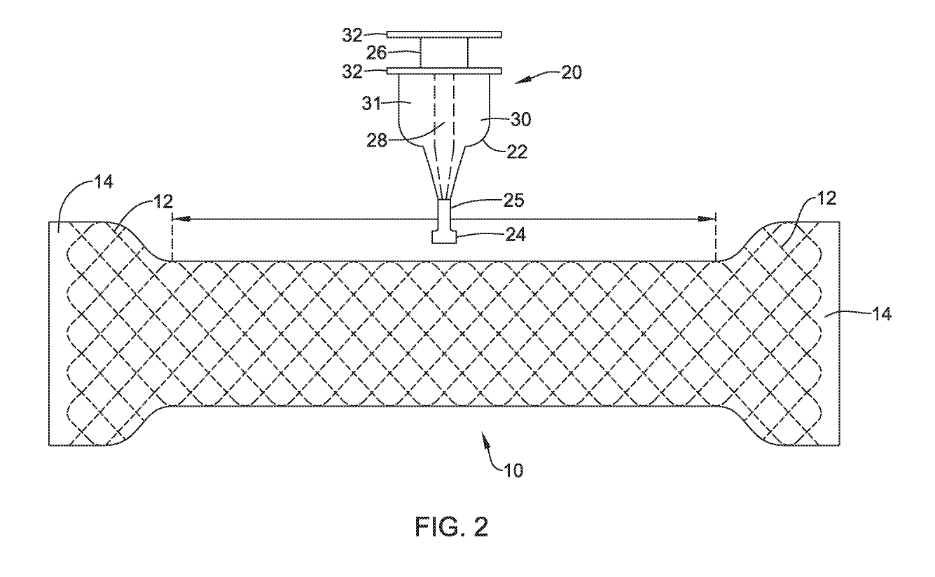

As discussed above, the stent 10 may be prepared with the radioactive coating 16 using a variety of techniques. For example, FIG. 2 illustrates one example method for preparing the radioactive stent 10. FIG. 2 is an illustration showing an applicator 20 placing a radioactive coating along all or a portion of the stent 10 (including filaments 12 and/or covering 14). As shown in FIG. 2, the applicator 20 may include a housing 22, a plunger 26, a mixing tube 25 and a tip member 24.

FIG. 2 further shows that the housing 22 of the applicator 20 may include a plurality of discrete chambers, such as a first housing chamber 28, a second housing chamber 30 and a third housing chamber 31. In some examples, the first housing chamber 28, second housing chamber 30 and third housing chamber 31 may be separated (e.g., discrete, closed off) from each other. FIG. 2 depicts separation walls within the housing 22 (depicted by the dashed lines) separating the first housing chamber 28, the second housing chamber 30 and the third housing chamber 31. While FIG. 2 shows the housing 22 including three separate chambers 28, 30 and 31, it is contemplated that the housing 22 may include less than or more than three chambers. For example, the housing 22 may include 1, 2, 3, 4, 5 or more chambers.

The housing 22 may include components of the radioactive coating 16 discussed above. For example, in some instances the housing 22 may include the radioactive material (e.g., microspheres) in the first housing chamber 28 and the substrate material (e.g., silicone) and/or an initiator (e.g., a curing agent) in one or more of the other chambers, such as the second and third housing chambers 30/31, respectively. In other words, the substrate material may be provided in or otherwise disposed in the second housing chamber 30, and the initiator may be provided in or otherwise disposed in the third chamber 31. Thus, the housing 22 may keep the components of the radioactive coating 16 separated from one another until it is desired to coat the stent 10 with the radioactive coating 16. However, it is also contemplated that applicator 20 may include a single housing portion 22 that includes a homogenous mixture of radioactive material and/or silicone and/or an initiator.

In can be appreciated that applicator 20 may be designed such that a clinician may apply the radioactive coating 16 along the stent 10 using one hand. For example, a clinician may grasp the example finger grips 32 with two fingers of a hand while applying pressure to the plunger 26 with the thumb of the same hand. The application of pressure to the plunger 26 may force the plunger 26 within the housing 22 such that the radioactive material is forced out of the tip 24 of the applicator 20. Therefore, a clinician may apply more or less pressure to dispose the desired amount of radioactive coating 12 along stent 10. Additionally, it is further contemplated that the length of the plunger 26 and/or housing 22 may vary to provide a described separation between the clinician and the radioactive material. For example, it can be appreciated that the applicator 20 shown in FIG. 2 may be designed with a long handle to reduce a clinician's exposure to radiation. For example, applicator 20 may include a housing 22 and/or plunger 26 that positions the grips 32 of the housing 22 5 cm or more, 10 cm or more, 15 cm or more, 20 cm or more, 25 cm or more, or 30 cm or more away from the chamber 28 containing the radioactive material. Thus, the clinician's hand used to apply the radioactive material may be kept away from the radioactive material by a distance of 5 cm or more, 10 cm or more, 15 cm or more, 20 cm or more, 25 cm or more, or 30 cm or more.

Further, the tip member (e.g., applicator tip) 24 may be designed such that the radioactive coating 16 may be wiped, painted, dabbed, spread, blotted, or otherwise applied onto selected portions of the stent 10. Additionally, in some examples, the tip member 24 may include a sponge material to aid in the application (or control the dispersion) of the radioactive material.

It can be further appreciated that the applicator 20 may be designed to mix two or more materials together prior to the application of the radioactive coating 16 onto the stent 10. For example, as discussed above, the housing 22 may include radioactive material (e.g., microspheres) in a first housing chamber 28 of the plurality of chambers, a substrate material 30 in a second housing chamber 30 of the plurality of chambers and an initiator material in a third housing chamber 31 of the plurality of chambers. Further, as the plunger 26 is driven into the housing 22 (including first chamber 28, second chamber 30 and third chamber 31), the radioactive material, substrate and/or initiator may be mixed together (e.g., combined) via a mixing zone prior to exiting the applicator 20 through the tip member 24. It is further contemplated that as the materials or components of the mixture (e.g., radioactive microspheres, silicone and/or initiator) are expelled from the plurality of chambers, they may be mixed in the mixing tube 25 connected to the housing 22.

In at least one example contemplated herein, the radioactive composition or mixture of the radioactive coating 16 described herein may include specific ratios of the radioactive microspheres, silicone and initiator. For example, in at least one example, the radioactive composition or mixture may include 5-10% of radioactive material and 90-95% of a combined silicone and initiator constituent. For example, one example may include 5% radioactive microspheres combined with 47.5% silicone and 47.5% initiator. However, other ratios of one or more of radioactive material, silicone and/or an initiator are contemplated.

The applicator 20 may be designed to mix two or more materials together, including the radioactive material, the substrate, and optionally an initiator, prior to the application of the radioactive coating 16 onto the stent 10 and just prior to the medical procedure in which the stent 10 will be implanted in the patient.

Additionally, the example applicator 20 shown in FIG. 2 may allow an operator flexibility as to when to apply the radioactive coating 16 to the stent 10. For example, in some instances the stent 10 may be prepared with the radioactive coating 16 within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient.

For example, in some instances the stent 10 may be prepared with the radioactive coating 16 at a medical treatment facility (e.g., hospital in which the stent is to be implanted into a patient), within a few hours prior to a stenting procedure at the medical treatment facility, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, medical personnel may be able to use the applicator 20 to apply the radioactive coating 16 just prior to insertion of stent 10 into the patient. In some instances, the applicator 20 may be used to apply the radioactive coating 16 after medical personnel places a medical order for the implantation of the stent in a patient's body. Further, the applicator 20 may allow medical personnel to customize the placement of the radioactive coating 16 on the stent 10 based on information analyzed within the surgical setting.

In other instances, the stent 10 may be prepared with the radioactive coating 16 at a radioactive material handling facility within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For purposes of this disclosure, a radioactive material handling facility may include a radiopharmacy, a licensed radioactive material distribution facility, or other licensed radioactive material handling facility. Further, it is contemplated that in some instances a medical treatment facility may request (e.g., order) a particular (e.g., custom designed) radioactive stent be prepared and delivered to a medical treatment facility for implantation into a patient within a few hours prior to a stenting procedure at the medical treatment facility. Additionally, it is contemplated that the stent 10 may be prepared with the radioactive coating 16 at a medical device manufacturing and/or distribution facility within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. Thus, the applicator 20 may be used to apply the radioactive coating 16 after medical personnel places a medical order for the implantation of the stent in a patient's body.

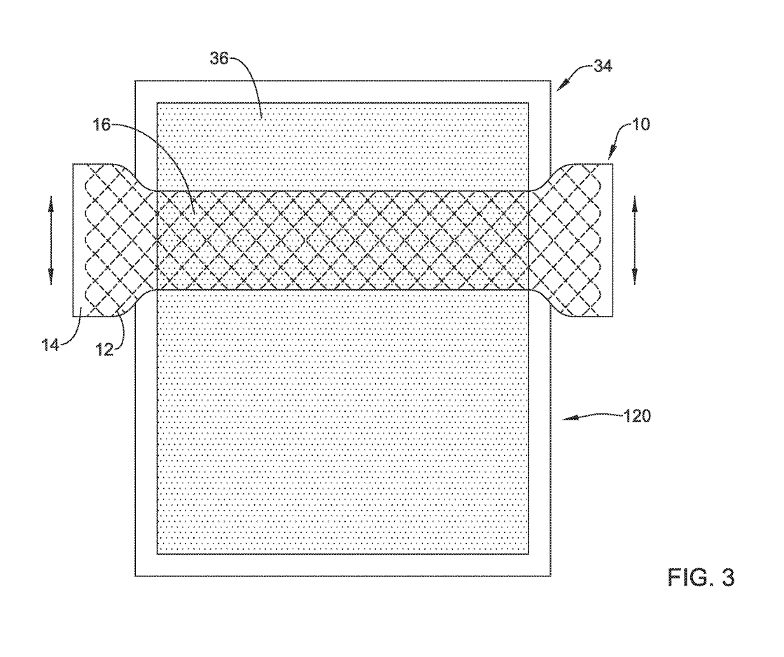

FIG. 3 illustrates another example method for preparing the radioactive stent 10 utilizing an applicator 120. The applicator 120 may include a base member 34 having a reservoir 36 for containing the radioactive mixture, including the mixture of the radioactive material (e.g., microspheres) and the substrate. FIG. 3 shows a method in which the stent 10 is rolled along the base member 34 having the reservoir 36. The reservoir 36 may include a radioactive mixture (e.g., microspheres mixed with a substrate) disposed therein. For example, the reservoir 36 may resemble a tray (e.g., bath) filled with a radioactive mixture that may be applied to the stent 10 as the stent 10 is rolled along the base member 34 such that the radioactive mixture contacts the stent 10 and is applied thereto to form the radioactive coating 16. In some instances, the height of the radioactive mixture in the reservoir 36 may be slightly raised above the base 34 such that the applicator 120 applies a “film” of the radioactive mixture to the stent 10. In other instances, the radioactive mixture may be “spread” across the base 34 (with or without a reservoir 36) prior to the stent 10 being rolled across the radioactive mixture. In yet other instances, the applicator 20 described in reference to FIG. 1 may be utilized to mix the radioactive material with a substrate and dispose the radioactive mixture in the reservoir 36 or otherwise apply the radioactive mixture to the base member 34, for example.

It can further be appreciated from FIG. 3 that the base member 34 (including the reservoir 36) may be sized such that the width of the base member 34 and reservoir 36 applies a desired amount of radioactive material onto the stent 10 as the stent 10 is rolled along the reservoir 36 (the rolling of the stent 10 along the reservoir 36 is depicted by the arrows shown in FIG. 3). The rolling action may apply a radioactive coating 16 uniformly around the circumference of the stent 10, such as a cylindrical body region of the stent 10. As in all examples discussed herein, this example method to apply radioactive coating 16 (e.g., microspheres) to the stent 10 may be utilized with a stent 10 including or not including a covering 14.

Additionally, methods used to prepare the radioactive stent 10 discussed above with respect to FIG. 3, or other embodiments disclosed herein, may coat the stent 10 very quickly. This is important because it limits the amount of time a clinician (or other person preparing the stent 10) is exposed to the radioactive material.

The example applicator 120 shown in FIG. 3 may allow an operator flexibility as to when to apply the radioactive coating 16 to the stent 10. For example, in some instances the stent 10 may be prepared with the radioactive coating 16 within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, in some instances the stent 10 may be prepared with the radioactive coating 16 similar to that described with respect to FIG. 2 above. For example, stent 10 may be prepared at a medical treatment facility or a radioactive material handling facility within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, in some instances an operator may be able to use the applicator 120 to apply the radioactive coating 16 just prior to insertion of stent 10 into the patient. In some instances, the applicator 120 may be used to apply the radioactive coating 16 after medical personnel places a medical order for the implantation of the stent in a patient's body. Further, the applicator 120 may allow an operator to customize the placement of the radioactive coating 16 on the stent 10 based on information analyzed within the surgical setting.

The two or more materials forming the radioactive mixture, including the radioactive material, the substrate, and optionally an initiator, may be mixed together prior to the application of the radioactive coating 16 onto the stent 10 at the medical treatment facility in which the stent 10 will be implanted in a patient or at a radioactive material handling facility and just prior to the medical procedure in which the stent 10 will be implanted in the patient.

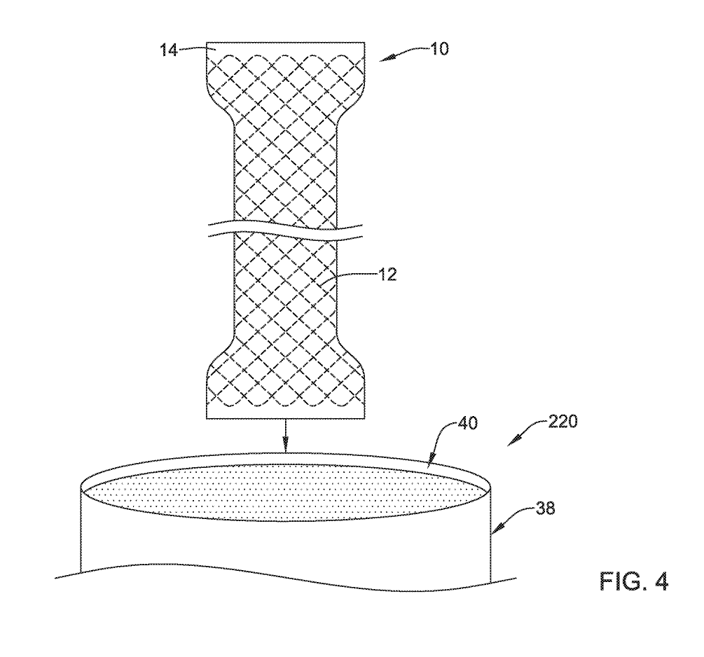

FIG. 4 illustrates another example process for preparing a radioactive stent 10. FIG. 4 is an illustration of utilizing a dip coating process to apply radioactive material (e.g., radioactive microspheres) to a stent 10 using an applicator 220. The applicator 220 may include a reservoir 38 including a radioactive mixture of a radioactive material and a substrate. As shown in FIG. 4, the stent 10 (including filaments 12 and/or covering 14) may be dipped (e.g., lowered) into the reservoir 38 to apply a radioactive coating to the stent 10. The reservoir 38 may include radioactive material (e.g., radioactive microspheres). The radioactive microspheres may be represented by the dot pattern shown in FIG. 4.

In at least some embodiments, dip coating the stent 10 with a radioactive mixture may occur while moving the stent 10 in and out of the radioactive mixture and/or rotating the stent 10 within the radioactive mixture. For example, the stent 10 may be coated by bringing the stent 10 into and out of the reservoir 38, rotating the stent 10 in the reservoir 38, or both. In some instances, however, rotation or translation may not be required. In some embodiments, the speed at which the stent 10 is translated and/or rotated may vary. In general, the rate of motion, duration of time in the reservoir 38 and/or cycles of submerging the stent 10 in the radioactive mixture may correlate to the amount of radioactive coating applied to stent 10. Further, it is also contemplated that any portion of the stent 10 may “masked” so that a portion of one or more structural characteristics (e.g., filaments 12 and/or covering 14) may be left unaltered or be altered to a lesser extent.

The example applicator 220 shown in FIG. 4 may allow an operator flexibility as to when to apply the radioactive coating 16 to the stent 10. For example, in some instances the stent 10 may be prepared with the radioactive coating 16 within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, in some instances the stent 10 may be prepared with the radioactive coating 16 similar to that described with respect to FIG. 2 above. For example, stent 10 may be prepared at a medical treatment facility or a radioactive material handling facility within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, in some instances an operator may be able to use the applicator 220 to apply the radioactive coating 16 just prior to insertion of stent 10 into the patient. In some instances, the applicator 220 may be used to apply the radioactive coating 16 after medical personnel places a medical order for the implantation of the stent in a patient's body. Further, the applicator 220 may allow an operator to customize the placement of the radioactive coating 16 on the stent 10 based on information analyzed within the surgical setting.

The two or more materials forming the radioactive mixture, including the radioactive material, the substrate, and optionally an initiator, may be mixed together prior to the application of the radioactive coating 16 onto the stent 10 at the medical treatment facility in which the stent 10 will be implanted in a patient or at a radioactive material handling facility and just prior to the medical procedure in which the stent 10 will be implanted in the patient.

In at least another example, it is contemplated that filaments 12 (of stent 10) may, themselves, be designed to contain radioactive material. For example, one or more filaments 12 may include a hollow portion designed to carry (e.g., hold, contain, etc.) radioactive material. In some examples, the center of one or more of filaments 12 may be hollow along the entire length of the filament. Further, it is contemplated that filaments 12 could be pre-injected with radioactive material prior to being formed (e.g., braided, wound, etc.) into stent member 10. In other examples, hollow filaments 12 could be injected with radioactive material just prior to stent 10 being delivered to a target site.

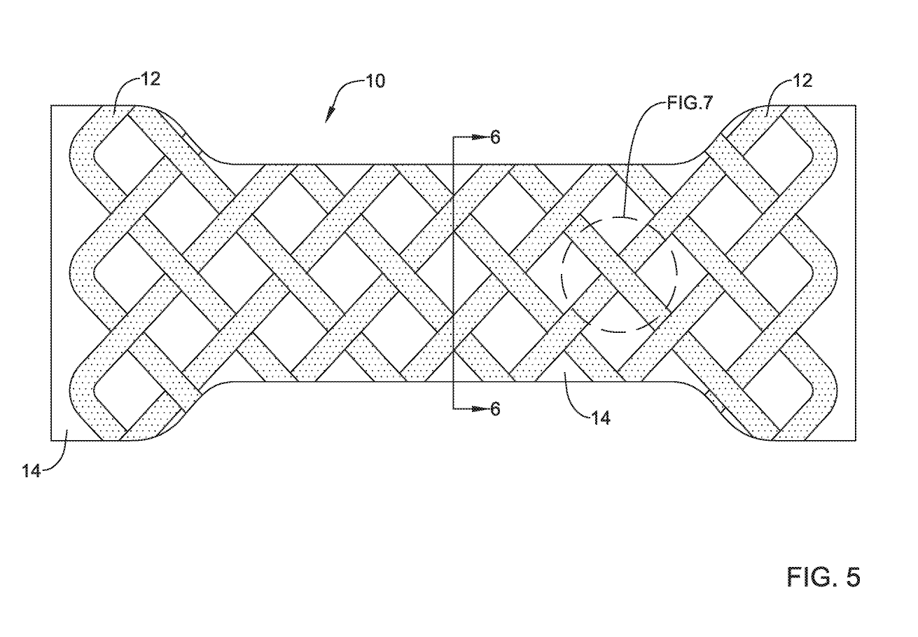

FIG. 5 shows another example stent 10 including filaments 12. As discussed above, the filaments 12 may include a radioactive material located within the filament 12 (e.g., within a hollow portion of the filament 12). FIG. 5 depicts the radioactive material located within filaments 12 by the dotted pattern within the filaments 12.

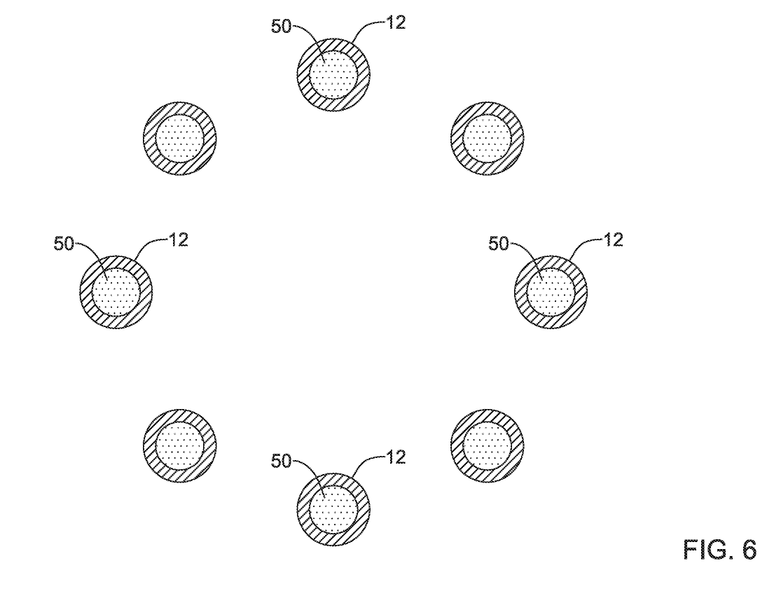

FIG. 6 is a cross-section along line 6-6 of the stent 10 shown in FIG. 5. As shown in FIG. 6, the cross-section of the individual filaments 12 of the stent 10 includes a radioactive mixture 50 including a radioactive material (depicted by the dotted pattern) positioned in the lumen of each individual hollow filament 12.

The two or more materials forming the radioactive mixture 50, including the radioactive material, the substrate, and optionally an initiator, may be mixed together prior to filling the lumens of the hollow filaments 12 of the stent 10 just prior to the medical procedure in which the stent 10 will be implanted in the patient. This may allow an operator flexibility as to when to apply the radioactive mixture 50 to the stent 10. For example, in some instances the stent 10 may be prepared with the radioactive mixture 50 within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. For example, in some instances the stent 10 may be prepared with the radioactive mixture 50 similar to that described with respect to FIG. 2 above. For example, stent 10 may be prepared at a medical treatment facility or a radioactive material handling facility within a few hours prior to a stenting procedure, such as less than 24 hours, less than 12 hours, less than 8 hours, less than 6 hours, less than 4 hours, less than 2 hours, or less than 1 hour prior to the surgical procedure in which the stent 10 is to be implanted into the patient. However, in other instances an operator may be able to use an applicator to apply the radioactive mixture 50 in the lumens of the hollow filaments 12 just prior to insertion of the stent 10 into the patient, such as after medical personnel places a medical order for the implantation of the stent in a patient's body. In some instances, this process may allow an operator to customize the placement of the radioactive mixture 50 in the lumens of the filaments 12 of the stent 10 based on information analyzed within the surgical setting.

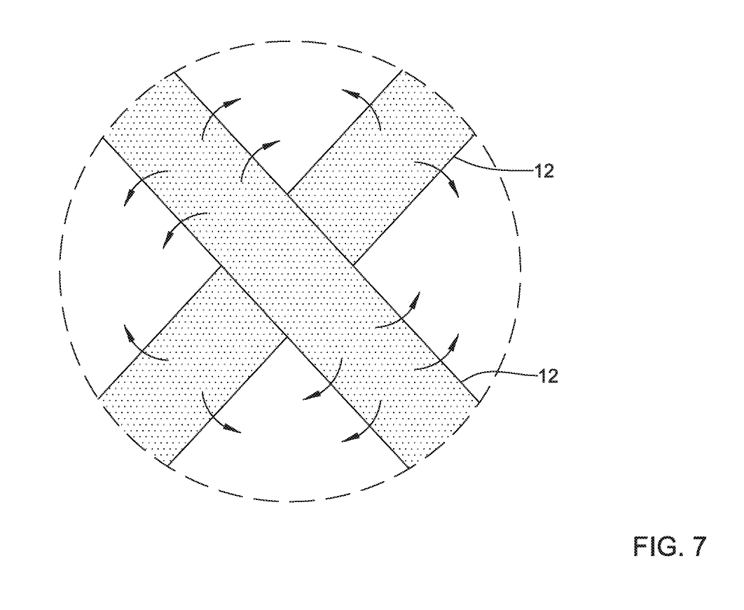

It is further contemplated that in at least the example discussed with respect to FIG. 5, a portion of the stent 10 may be designed to elute the radioactive material from a position in the hollow portion of the filament 12 to a position outside of the filament 12 (e.g., to target tissue adjacent the filament 12). In other words, one or more of filaments 12 may be designed with a given porosity that permits the radioactive material positioned in a hollow portion of the filament 12 to elute through the wall of the filament 12 surrounding the radioactive mixture 50. FIG. 7 illustrates the radioactive material (depicted by the dotted pattern in FIG. 7) eluting from the lumen of the filament 12 to outside (and adjacent to) the filaments 12.

It can be appreciated that the porosity of the filament 12 may be designed such that the rate of radioactive material that is released may be controlled. For example, the porosity of the filament 12 may be configured such that the radioactive material is slowly released, quickly released, or the like. Further, different portions of the filament 12 may release radioactive material at different rates.

In at least one example, a method of constructing a stent 10 including a radioactive material located within at least one hollow filament 12 may include first forming a stent 10 by braiding or otherwise forming a mesh of one or more hollow, porous filaments 12. Next, the braided or interwoven mesh of hollow, porous filaments 12 may be bound together by a covering 14. Further, a radioactive mixture including a radioactive material and a substrate may then be injected into the hollow portion of the filaments 12. It can then be appreciated that the radioactive material may elute out of the porous filament 12 over a duration of time. Further, in some examples the radioactive mixture may further disperse the radioactive material in a uniform manner, thereby, creating an evenly dispersed radioactive “footprint” across the stent 10.

In addition, it can be appreciated that at least a portion of the filament 12 may include a biodegradable material. Therefore, it is contemplated that the portion of a filament 12 which includes a biodegradable material may degrade over a pre-determined time period, thereby revealing the radioactive material underneath the biodegradable portion. Additionally, it is contemplated that the stent 10 may include a combination of porous portions and/or biodegradable portions.

Materials that may be used for the various components of the stent 10 and the various examples disclosed herein may include those commonly associated with medical devices. For simplicity purposes, the disclosure makes reference to a stent 10. However, this is not intended to limit the devices and methods described herein, as the discussion may be applied to other similar systems and/or components of stent systems or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A method of preparing a stent, the method comprising:
applying a coating to a portion of the stent less than 24 hours prior to implanting the stent into a patient, the coating including:
a plurality of radioactive elements; and
a substrate;
wherein the plurality of radioactive elements are mixed with the substrate to form a mixture such that the plurality of radioactive elements are dispersed within the substrate prior to the coating being applied on the stent.

2. The method of claim 1, wherein the plurality of radioactive elements include a plurality of microspheres.

3. The method of claim 1, wherein the radioactive elements are selected from the group comprising: Iodine-125, Cesium-13, Palladium-103, Yttrium-90 and Holmium-166.

4. The method of claim 1, wherein the substrate includes silicone.

5. The method of claim 1, wherein the half-life of the radioactive elements is less than or equal to 60 days.

6. The method of claim 1, wherein applying the coating to a portion of the stent includes placing the radioactive elements in a first chamber of an applicator, placing the substrate in a second chamber of the applicator, and utilizing the applicator to mix the radioactive elements and the substrate prior to applying the coating to the stent.

7. The method of claim 1, wherein applying the coating to a portion of the stent further includes placing the mixture of the radioactive elements and the substrate on a base member, and thereafter rolling the stent on the base member such that the mixture is applied to the stent.

8. The method of claim 1, wherein applying the coating to a portion of the stent includes placing the mixture of radioactive elements and the substrate into a reservoir, and thereafter dipping the stent into the reservoir such that the mixture is applied to the stent.

9. An applicator for applying a radioactive coating on a stent, comprising:
a housing including a plurality of discrete chambers;
a mixing tube, the mixing tube having a first end and a second end, wherein the first end is connected to the one of more chambers of the housing; and
a tip member, the tip member connected to the second end of the mixing tube;
wherein a first one of the plurality of chambers of the housing is configured to contain a plurality of radioactive elements;
wherein at least a second one of the plurality of chambers of the housing is configured to contain a substrate;
wherein the plurality of radioactive elements and the substrate are mixed in the applicator prior to being applied to the stent.

10. The applicator of claim 9, wherein the plurality of radioactive elements includes at least one microsphere.

11. The applicator of claim 9, wherein the substrate includes silicone.

12. The method of claim 9, wherein the radioactive elements are selected from the group comprising: Iodine-125, Cesium-13, Palladium-103, Yttrium-90 and Holmium-166.

13. The method of claim 9, wherein the plurality of chambers further includes a third chamber containing an initiator, wherein the initiator is configured to cure the substrate.

14. The method of claim 9, wherein the half-life of the radioactive elements is less than or equal to 60 days.

15. A kit for preparing a radioactive stent at a medical treatment facility, the kit comprising:
a stent;
an applicator including a housing, the housing including a plurality of discrete chambers;
wherein a first one of the plurality of discrete chambers of the applicator is configured to contain a radioactive material;
wherein the applicator is configured to apply the radioactive material to the stent prior to implanting the stent within a patient.

16. The kit of claim 15, wherein the radioactive material includes a plurality of radioactive microspheres.

17. The kit of claim 16, wherein the plurality of discrete chambers of the housing further includes a second chamber and a third chamber, wherein the first chamber includes the plurality of radioactive microspheres, the second chamber includes a silicone and the third chamber includes an initiator designed to cure the silicone.

18. The kit of claim 17, wherein the applicator is configured to mix the plurality of radioactive microspheres, the silicone and the initiator prior to applying the radioactive material to the stent.

19. The kit of claim 18, wherein the radioactive material is applied to the stent such that the plurality of microspheres are uniformly distributed on the stent.

20. The kit of claim 19, wherein the half-life of the plurality of microspheres is less than or equal to 60 days.
